(19) United States
(12) Patent Application Publication
(10) Pub. No.: US 2025/0208063 A1
(43) Pub. Date: Jun. 26, 2025

(54) RADIO/MICROWAVE FREQUENCY SOLID CONSTITUENT SENSOR

(71) Applicant: Know Labs, Inc., Seattle, WA (US)

(72) Inventors: Barry SHELTON, Seattle, WA (US); Dominic KLYVE, Seattle, WA (US)

(21) Appl. No.: 18/395,878

(22) Filed: Dec. 26, 2023

(51) Int. Cl. G01N 22/00; G01N 33/2022; G01N 33/24; G01N 33/38

(52) U.S. Cl. CPC G01N 22/00; G01N 33/2022; G01N 33/241; G01N 33/383

(57) ABSTRACT

A solid constituent sensor or sensor system that uses one or more radio frequency (RF) or microwave frequency signals transmitted to and/or into a solid sample to aid in making a determination about the solid sample. This is generally referred to as characterizing a solid sample. Characterizing the solid sample can include: detecting one or more constituents in the sample; detecting the lack of one or more constituents in the sample; determining the molecular makeup of homogenous or heterogeneous solid sample; determining whether the solid sample has an expected chemical composition; determining whether the solid sample deviates from an expected chemical composition; identifying the solid sample; and others.

### FIELD

This disclosure relates generally to apparatus, systems and methods of characterizing a solid sample using a sensor that operates in the radio or microwave frequency range of the electromagnetic spectrum.

### BACKGROUND

A number of products exist that detect or otherwise characterize solid materials. For example, X-ray fluorescence sensors and commercially-available metal detectors are known that can detect some characteristics of homogenous and heterogeneous solids using electromagnetic (EM) radiation in their extremely high and low frequency bands, respectively. The ionizing nature of X-ray radiation may make it unsuitable for everyday civilian use. In addition, nuclear quadrupole resonance (NQR) sensors provide a method of finding the EM resonance of a solid constituent, and the sensors used in NOR typically require a cylindrical or conical physical sample structure to be placed in a coil.

### SUMMARY

A solid constituent sensor or sensor system is described herein that uses signals in the radio frequency/microwave frequency range. The solid constituent sensor and methods described herein use one or more radio or microwave frequency signals transmitted to and/or into a solid sample to aid in making a determination about the solid sample. This is generally referred to as characterizing a solid sample. Characterizing the solid sample can include, but is not limited to: detecting one or more constituents in the solid sample; detecting the lack of one or more constituents in the solid sample; determining the molecular makeup or chemical composition of homogenous or heterogeneous solid constituents; determining whether the solid sample has an expected chemical composition; determining whether the solid sample deviates from an expected chemical composition; determining whether the solid sample has certain types of structures, microstructures or defects; identifying the solid sample; and others.

A solid constituent sensor or sensor system described herein can include at least one transmit antenna and at least one receive antenna. The at least one transmit antenna is positioned and arranged to transmit a transmit signal to and/or into a solid sample, and the at least one receive antenna is positioned and arranged to detect a response resulting from transmission of the transmit signal by the at least one transmit antenna to and/or into the solid sample. A transmit circuit is electrically connectable to the at least one transmit antenna, and the transmit circuit is configured to generate the transmit signal in a radio or microwave frequency range of an electromagnetic spectrum. A receive circuit is electrically connectable to the at least one receive antenna, and the receive circuit is configured to receive the response detected by the at least one receive antenna. A processor in communication with the receive circuit is configured to execute instructions to characterize the solid sample based on the detected response.

In operation, the signal detected by the receive antenna that results from transmitting the signal by the transmit antenna can be analyzed (e.g., processed and/or compared with at least one reference signal) to make a determination about the solid sample. The comparison can take many forms. For example, the comparison can be whether the detected signal matches the reference signal. For example, the comparison can be whether the detected signal deviates from the reference signal, for example by a predetermined amount.

A solid constituent characterization method can include transmitting a transmit signal from a transmit antenna to and/or into a solid sample, where the transmit signal is in a radio frequency (RF) or microwave frequency range of the electromagnetic spectrum. A response signal that results from transmission of the transmit signal to and/or into the solid sample is detected at a receive antenna. The detected response signal (or a derivative thereof) is then used to characterize the solid sample.

### DRAWINGS

FIG. 1 is a schematic depiction of a non-invasive solid constituent sensor system with a non-invasive solid constituent sensor relative to a solid sample, according to an embodiment described herein.

FIG. 2 illustrates an example of an antenna array described herein that can be used in a non-invasive solid constituent sensor or sensor system, according to an embodiment described herein.

FIG. 3 depicts illustrates another example of an antenna array described herein that can be used in a non-invasive solid constituent sensor or sensor system, according to an embodiment described herein.

FIG. 4 depicts a signal storage, according to an embodiment described herein.

FIG. 5 depicts a flow diagram of a solid constituent characterization method, according to an embodiment described herein.

FIG. 6 depicts RF spectrums of different metal objects recorded by the sensor described herein across a range of frequencies.

### DETAILED DESCRIPTION

The following is a detailed description of apparatus, systems and methods of characterizing a solid sample via spectroscopic techniques using frequencies in the radio or microwave frequency bands of the electromagnetic spectrum. A solid constituent sensor described herein includes at least one antenna which functions as a transmit antenna to transmit an electromagnetic signal in the radio or microwave frequency range to and/or into a solid sample and at least one antenna which functions as a receive antenna that receives/detects an electromagnetic signal in the radio or microwave frequency range resulting from transmission of the electromagnetic signal to and/or into the solid sample. The signal(s) detected by the receive antenna(s) is then used to characterize the solid sample, for example by comparing the detected signal or a derivative thereof to one or more stored reference signals.

Based on the characterization, a notification/warning/alert signal can be generated and transmitted to a notification/warning/alert device. The notification/warning/alert signal can inform a user the identify or status of the detected solid sample, or alert a user to a problem with the solid sample. The notification/warning/alert signal can alert a user that the solid constituent material is as expected, and therefore the solid sample including the solid constituent material is at an expected status. The notification/warning/alert signal can notify or alert a user that the solid sample is trending over time toward a potential problem. The notification/warning/alert signal can alert a user that the solid sample has been stable (i.e., not changing or changing within a predetermined range) over a time period. The notification/warning/alert signal device can generate a visual notice, such as a light or text on a display screen, an audible notice, a haptic notice, or the like.

Examples of sensors that can detect constituents using radio frequency or microwave frequency signals are described in U.S. Pat. Nos. 10,548,503, 11,063,373, 11,058,331, 11,033,208, 11,284,819, 11,284,820, 10,548,503, 11,234,619, 11,031,970, 11,223,383, 11,058,317, 11,193,923, 11,234,618, 11,389,091, U.S. 2021/0259571, U.S. 2022/0077918, U.S. 2022/0071527, U.S. 2022/0074870, U.S. 2022/0151553, each of which is incorporated herein by reference in its entirety.

As referenced herein, a “solid sample” may refer to a physical portion or specimen of a solid material that can be analyzed or characterized for its structures, chemical compositions, characteristics, properties (e.g., physical/chemical/mechanical/electrical properties), identification of the sample, etc. Example solid samples described herein may include, but are not limited to, metals (e.g., irons), alloys (e.g., iron compounds), minerals, polymers, ceramics, etc. For example, an iron compound sample can be analyzed or characterized to determine the carbon content thereof, which may be used to determine a number of related physical properties of the iron compound including, for example, brittleness, hardness, corrosion-resistance, etc.

As referenced herein, an “constituent” or “constituents” contained in a solid sample can be any constituent that one may wish to detect. The constituents may vary widely depending on the type of solid sample and/or the analytical and characterization objectives. In an example, for a solid metal or metal alloy sample (e.g., an iron compound), suitable constituents may include, but are not limited to, a chemical composition such as a carbon content, an impurity, a trace element, a structure, a defect, a microstructure, a water content, a water-induced corrosion product, etc. In another example, for a solid concrete sample, suitable constituents may include, but are not limited to, various concrete constituents such as a cement content, an aggregate (e.g., a sand, a gravel, a crushed stone, etc.), an admixture (e.g., a chemical additive such as an accelerator, retarder, a superplasticizer, etc.), an air-entraining agent, an air void, a reinforcing material, a cementitious material, a moisture content, etc. In yet another example, for a soil sample, suitable constituents may include, but are not limited to, various soil constituents such as a moisture content, a nutrient (e.g., nitrogen, phosphorus, potassium, etc.), a particular type of cations (i.e., positively charged ions), an organic matter content, a salt content, a soil texture (e.g., proportions of sand, silt, and clay), a heavy metal (e.g., lead, cadmium, etc.), a contaminant (e.g., a pesticide, a pollutant, etc.), a microbial population, a biological activity, a mineral composition, a particle size distribution, etc. In yet another example, for a plant sample, suitable constituents may include, but are not limited to, a chemical composition such as plant-specific compounds (e.g., alkaloids, flavonoids, terpenoids, etc.), secondary metabolites (e.g., tannins, phenolics, etc.), nutrients (e.g., nitrogen, phosphorus, potassium, etc.), a mineral content, a tissue structure, a water content, etc.

As referenced herein, “homogeneous solid constituent” or “heterogeneous solid constituent” may refer to the uniformity or non-uniformity of the composition and/or properties within a solid sample regarding one or more constituents to be detected. In a homogeneous solid sample, the composition and properties of a constituent may be substantially consistent and uniform throughout the entire sample. In a heterogeneous solid sample, there may be regions or phases with distinct compositions, structures, or properties. For example, a granite rock can be a heterogeneous solid sample since it may include different minerals at different regions.

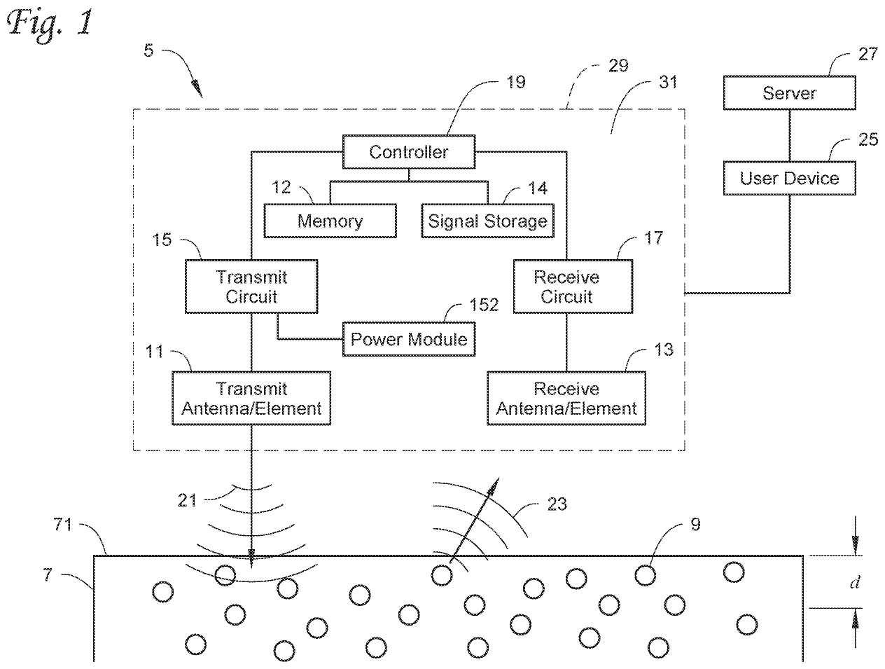

Referring now to FIG. 1, an embodiment of a solid constituent sensor system with a solid constituent sensor system 5 is illustrated. The sensor system 5 is depicted relative to a solid sample 7 that contains a constituent of interest 9, for example a chemical constitute within the solid sample. In this example, the sensor system 5 is depicted as including an antenna array that includes a transmit antenna/element 11 (hereinafter “transmit antenna 11”) and a receive antenna/element 13 (hereinafter “receive antenna 13”). The sensor system 5 further includes a transmit circuit 15, a receive circuit 17, and a controller 19. As discussed further below, the sensor system 5 can also include a power supply, such as a battery (not shown in FIG. 1). In some embodiments, power can be provided from mains power, for example by plugging the sensor system 5 into a wall socket via a cord connected to the sensor system 5.

The transmit antenna 11 is positioned, arranged and configured to transmit a signal 21 that is the radio frequency (RF) or microwave frequency range of the electromagnetic spectrum to or into a surface 71 of the solid sample 7. The transmit antenna 11 can be an electrode or any other suitable transmitter of electromagnetic signals in the radio frequency (RF) or microwave frequency range. The transmit antenna 11 can have any arrangement and orientation relative to the solid sample 7 that is sufficient to allow the sample sensing, detecting, or characterization to take place. In one non-limiting embodiment, the transmit antenna 11 can be arranged to face in a direction that is substantially toward the surface 71 of the solid sample 7.

In an embodiment, the sensor system 5 or a portion of the sensor system 5 can be located exterior of the solid sample 7 during sample characterization. For example, the transmit antenna 11 and the receive antenna 13 may be located exterior of the solid sample 7 and in proximity to the surface 71. In an embodiment, the sensor system 5 or a portion of the sensor system 5 may be located inside the solid sample 7 during sample characterization. For example, the transmit antenna 11 and the receive antenna 13 may be located within a hollow space or cavity of the solid sample 7 and in proximity to an internal surface of the solid sample 7. In an embodiment, a portion of the sensor system 5 (e.g., the transmit antenna 11 and the receive antenna 13) can be mounted to a housing (e.g., a probe head) which is configured to accommodate the antennas and provide mechanical support and protection. The probe head may insert into a solid sample (e.g., a soil sample) during sample characterization.

In an embodiment, a coupling medium or mechanism may be provided between the transmit and receive antennas 11, 13 and the solid sample 7 to facilitate the transfer of radio frequency (RF) or microwave frequency energy between the sensor system 5 and the solid sample 7. The coupling medium may include, but is not limited to, a dielectric material, a matching fluid, a waveguide, or the like. The coupling medium or mechanism may be flexible and have any suitable configurations.

The signal 21 transmitted by the transmit antenna 11 is generated by the transmit circuit 15 which is electrically connectable to the transmit antenna 11. The transmit circuit 15 can have any configuration that is suitable to generate a transmit signal to be transmitted by the transmit antenna 11. Transmit circuits for generating transmit signals in the RF or microwave frequency range are well known in the art. In an embodiment, the transmit circuit 15 can include, for example, a connection to a power source, a frequency generator, and optionally filters, amplifiers, frequency control mechanisms, or any other suitable elements for a circuit generating an RF or microwave frequency electromagnetic signal. In an embodiment, the signal generated by the transmit circuit 15 can have a frequency that is in the range from about 10 kHz to about 100 GHz. In another embodiment, the frequency can be in a range from about 300 MHz to about 6000 MHz. In another embodiment, the frequency range can be from about 10 kHz to about 10 GHz. In an embodiment, the transmit circuit 15 can be configured to sweep through a range of frequencies that are within the range of about 10 kHz to about 100 GHz, or in another embodiment a range of about 300 MHZ to about 6000 MHz.

In the embodiment depicted in FIG. 1, the sensor system 5 further includes a power module 152 to boost the power of the signal generated by the transmit circuit 15. It is to be understood that the power module 152 may be integrated with the transmit circuit 15. The power module 152 may include, for example, a power amplifier to take a relatively low-power signal from an RF/microwave signal generator of the transmit circuit 15 and amplify the power to a higher level. A power amplifier with appropriate gain and power handling capabilities can be used to achieve the desired power level. Example power amplifiers may include, but are not limited to, a solid-state amplifier, a traveling-wave tube amplifier, or the like. In an embodiment, the power module 152 may be adjustable to boost the power of the signal generated by the transmit circuit 15 from the original level to different higher levels (e.g., a first level and a second level higher than the first level). It is to be understood that the power module 152 may boost the power continuously or to discrete higher levels. The power module 152 may further include control and monitoring mechanisms to allow a user, via the controller 19, to adjust the power level, monitor temperature, and protect against overdrive or other malfunctions.

In an embodiment, the transmit antenna 11 may be controlled to switch between multiple sets of antenna(s) to handle the different levels of increased power boosted by the power module 152. For example, the transmit antenna 11 may be controlled to switch between a first set of antenna(s) corresponding to the first level of boosted power, and a second set of antenna(s) corresponding to the second, higher level of boosted power.

In an embodiment, the transmit signal 21 may be adjusted, via one or more of the transmit circuit 15, the power module 152, and the transmit antenna 11, to adjust a penetration depth d into the solid sample 7. For example, the power module 152 may be controlled to boost the power of the transmit signal 21 to increase the penetration depth d. The transmit circuit 15 may be controlled to adjust the frequency of the transmit signal 21 to adjust the penetration depth d. It is to be understood that the depth of penetration into different solid materials may depend on various factors, including, for example, the signal frequency, the solid material properties, the signal wavelength/frequency, the signal power, etc. For example, the penetration depth may vary from a few micrometers, to a few millimeters, to a few centimeters, or to a few meters, depending on the solid material properties. In an embodiment, the penetration depth is less than one centimeter. The controller 19 may control the respective parts of the sensor system 5 to adjust the penetration depth suitable for desired applications. In another embodiment, the penetration depth d may be close to zero (e.g., a few micrometers or less, or a few nanometers or less) in which case the solid sample is characterized based on a signal reflected back from the surface 71.

The receive antenna 13 is positioned, arranged, and configured to detect one or more electromagnetic response signals 23 that result from the transmission of the transmit signal 21 by the transmit antenna 11 to and/or into the solid sample 7 and impinging on the constituent 9 or a local area including the constituent 9. The receive antenna 13 can be an electrode or any other suitable receiver of electromagnetic signals in the radio frequency (RF) or microwave range. In an embodiment, the receive antenna 13 is configured to detect an electromagnetic signal having a frequency that is in the range from about 10 kHz to about 100 GHz, or in another embodiment a range from about 300 MHz to about 6000 MHz. In another embodiment, the frequency can be in a range from about 10 kHz to about 10 GHz. The receive antenna 13 can have any arrangement and orientation relative to the solid sample 7 that is sufficient to allow detection of the response signal(s) 23 to allow the characterization to take place. In one non-limiting embodiment, the receive antenna 13 can be arranged to face in a direction that is substantially toward the surface 71 of the solid sample 7.

The receive circuit 17 is electrically connectable to the receive antenna 13 to convert the electromagnetic energy detected by the receive antenna 13 into one or more signals reflective of the response detected by the receive antenna 13. The receive circuit 17 can have any configuration that is suitable for interfacing with the receive antenna 13 to convert the electromagnetic energy detected by the receive antenna 13 into one or more signals reflective of the response signal(s) 23. The construction of receive circuits are well known in the art. The receive circuit 17 can be configured to condition the signal(s) prior to providing the signal(s) to the controller 19, for example through amplifying the signal(s), filtering the signal(s), or the like. Accordingly, the receive circuit 17 may include filters, amplifiers, or any other suitable components for conditioning the signal(s) provided to the controller 19. Further information on a transmit circuit and a receive circuit in an analyte sensor is disclosed in U.S. Pat. No. 11,063,373 the entire contents of which are incorporated herein by reference.

In an embodiment, the receive circuit 17 may include an analog to digital (AD) converter to convert the signal detected by the receive circuit 17 from an analog signal into a digital processor readable format. The AD converter may be separate from the receive circuit 17 or the AD converter may be considered part of the receive circuit 17. Similarly, a digital to analog converter, which may be part of or separate from the transmit circuit 15, converts a digital signal into an analog signal for transmission by the transmit antenna 11.

The controller 19 controls operation of the sensor system 5, including controlling the transmit circuit 15 and the receive circuit 17. The controller 19 may include a processor, also known as a central processing unit (CPU), which may facilitate the operation of the sensor system 5 according to instructions stored in memory 12. The processor may include suitable logic, circuitry, interfaces, and/or code that may be configured to execute a set of instructions stored in the memory 12. The processor may be a hardware component that performs arithmetic, logic, and control operations on data. The processor may be comprised of the arithmetic logic unit, control unit, memory subsystems, and other subsystems. The processor may be responsible for performing signal procession and/or comparison, as well as arithmetic and logical operations on data. The processor may include components for addition, subtraction, multiplication, and division and logical operations such as AND, OR, and NOT. The processor may be responsible for fetching instructions from the memory 12, decoding them, and executing them. The processor may manage the flow of data between different components of the solid constituent sensor as a whole, ensuring that operations are performed in the correct order, and that data is transferred efficiently. The processor may provide fast access to frequently used data and instructions. The processor may include components such as caches, registers, and pipelines, which are designed to minimize the time required to access and manipulate data. The processor may include various other components and subsystems, such as instruction set architecture (ISA), which may define the set of instructions that the processor can execute. The processor may specify the format of instructions and data, the addressing modes used to access memory 12 and I/O devices, and the interrupt and exception handling mechanisms used to manage errors and other events. The processor may include advanced instruction execution capabilities, support for virtualization and parallel processing, and power management mechanisms that reduce energy consumption and heat dissipation. In some embodiments, the controller 19 may be a microcontroller, programmable logic controller (PLCs), or digital signal processors (DSPs) and may be implemented as software components running on general-purpose computers or embedded systems.

A signal storage 14 stores one or more reference signals. The signal storage 14 may be any form of storage that is suitable for storing signals of reference solid samples for use in analyzing (e.g., comparing) the signal detected by the receive antenna 13 to detect and characterize the solid sample. For example, the signal storage 14 may store a database containing signal data related to the reference signals obtained from reference solid samples. The signals may present in any suitable forms, such as, for example, waveforms, spectroscopy diagrams (e.g., spectroscopy lines), frequency spectrum, time-domain signals, amplitude and/or phase signals, imaging signals, modulated signals, etc. The signal storage 14 may be part of the memory 12 or may be at a location separate from the memory 12.

The reference signals stored in the signal storage 14 can be retrieved by, e.g., the processor of the controller 19, and used to analyze the response signal detected by the receive circuit 17. In an embodiment, one or both of the response signal from the receive circuit 17 and the reference signal retrieved from the signal storage 14 can be processed by the controller 19 and transformed to any suitable format that can be compared to each other.

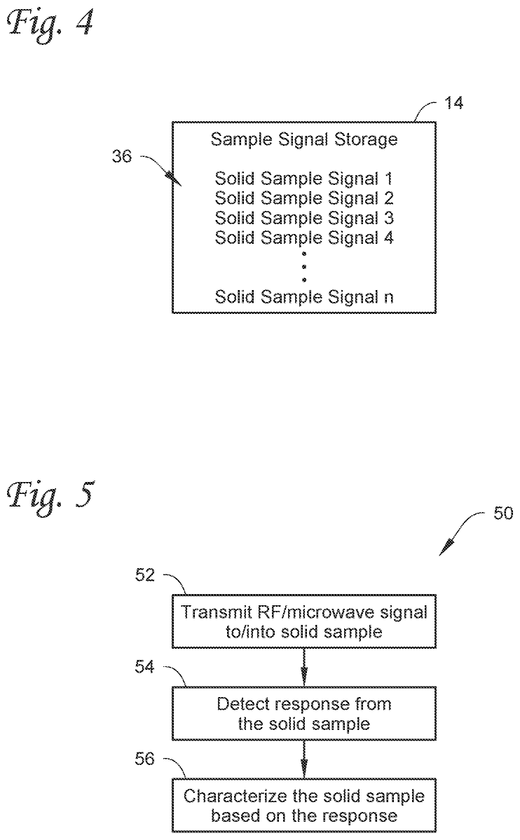

For example, the controller 19 can perform a signal waveform comparison between the signal from the receive circuit 17 and the reference signal to detect or characterize the solid sample 7. The techniques for comparison of signal waveforms are well-known in the art. The signal waveform comparison may be to match the detected signal waveform with the stored reference signal(s) 36 (see FIG. 4). This may assist in detecting a particular constituent in the solid sample; detecting a lack of a particular constituent in the solid sample; determining the molecular makeup of homogenous or heterogeneous solid constituents; determining the total makeup of a solid sample with or without suspended solids; determining the total amount of a combination of multiple constituents in a solid sample; determining whether the solid sample has an expected makeup; identifying the solid sample; etc. The signal comparison may be to determine when the detected signal waveform deviates from the stored reference signal 36 by a predetermined amount. For example, for a detected iron compound sample, when the detected signal waveform deviates by 10% or more (or 3% or more; or 5% or more; etc.) from the stored reference signal 36, that may indicate the iron compound sample has a carbon content deviating from a predetermined content range. Such a deviation of carbon content from the predetermined content range may indicate that the iron compound sample has a problem such as, for example, metal fatigue in terms of its brittleness, hardness, corrosion resistance, etc.

In one embodiment, the controller 19 may optionally be in communication with at least one external device 25 such as a user device and/or a remote server 27, for example through one or more wireless connections such as Bluetooth, wireless data connections such a 4G, 5G, LTE or the like, or Wi-Fi. If provided, the external device 25 and/or remote server 27 may process (or further process) the signals that the controller 19 receives from the receive circuit 17, for example to characterize the solid sample 7. If provided, the external device 25 may be used to provide communication between the sensor system 5 and the remote server 27, for example using a wired data connection or via a wireless data connection or Wi-Fi of the external device 25 to provide the connection to the remote server 27.

With continued reference to FIG. 1, the sensor system 5 may include a sensor housing 29 (shown in dashed lines) that defines an interior space 31. Components of the sensor system 5 may be attached to and/or disposed within the housing 29. For example, the transmit antenna 11 and the receive antenna 13 are attached to the housing 29. In some embodiments, the antennas 11, 13 may be entirely or partially within the interior space 31 of the housing 29. In some embodiments, the antennas 11, 13 may be attached to the housing 29 but at least partially or fully located outside the interior space 31. In some embodiments, the transmit circuit 15, the receive circuit 17 and the controller 19 are attached to the housing 29 and disposed entirely within the sensor housing 29.

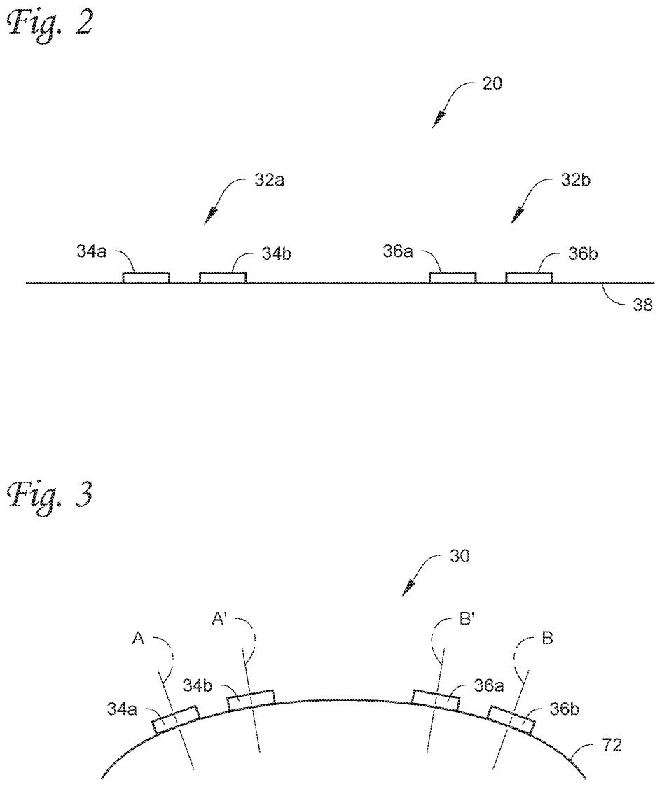

FIG. 2 illustrates an example of an antenna array 20 that can be used in the sensor system 5 in place of the antennas 11, 13. The antenna array 20 is depicted as having a first antenna set 32a and a second antenna set 32b. The first antenna set 32a includes a first antenna 34a and a second antenna 34b, and the second antenna set 32b includes a third antenna 36a and a fourth antenna 36b. The first antenna 34a, the second antenna 34b, the third antenna 36a, and the fourth antenna 36b may each include an elongated strip of conductive material with a longitudinal axis (e.g., toward or out of the plane of the paper). The longitudinal axes of the antennas 34a, 34b, 36a, 36b may be parallel to one another, substantially parallel to one another, or generally parallel to one another with a slight angle between one or more of the longitudinal axes of one or more of the antennas 34a, 34b, 36a, 36b. In the embodiment depicted in FIG. 2, the antennas 34a, 34b, 36a, 36b are disposed on a common substrate 38 which is substantially planar as shown in FIG. 2. It is to be understood that the antennas 34a, 34b, 36a, 36b may be disposed on separate planar substrates, angled substrate(s), curved substrate(s), or other substrates. The antennas 34a, 34b, 36a, 36b are depicted in FIG. 2 as disposed on and projecting above the surface of the substrate 38. It is to be understood that the antennas 34a, 34b, 36a, 36b can have other arrangements relative to the substrate such as being flush with the surface of the substrate 38. The substrate 38 may be formed in a manner so it does not interfere with operation of the antenna array 20 as transmit/receive antenna, for example being formed of a non-conductive material such as plastic.

In an embodiment, any one or more of the antennas 34a, 34b, 36a, 36b can be connected to the transmit circuit 15 whereby any one or more of the antennas 34a, 34b, 36a, 36b can function as a transmit antenna. In addition, any one or more of the antennas 34a, 34b, 36a, 36b can be connected to the receive circuit 17 whereby any one or more of the antennas 34a, 34b, 36a, 36b can function as a receive antenna to detect a response that results from transmitting the transmit signal to and/or into the solid sample. Controlling any of a number of antennas to transmit an RF signal and any of a number of antennas to receive an RF signal is disclosed in U.S. Pat. Nos. 11,058,331, 11,193,923, and 11,330,997 each one of which is incorporated herein by reference in its entirety.

The embodiment depicted in FIG. 2 is illustrated as including four antennas, with each antenna set 32a, 32b of the antenna array 20 including two antennas. It is to be understood that a larger or smaller number of antennas can be used.

FIG. 3 illustrates an example of an antenna array 30 that can be used in the sensor system 5 in place of the antennas 11, 13. In FIG. 3, elements that are the same as or similar to elements in FIG. 2 are referenced using the same reference numerals. In this embodiment, the antennas 34a, 34b, 36a, 36b are disposed on a curved or arcuate common substrate 72. The antennas 34a, 34b, 36a, 36b can have the same configuration as the antennas described above for FIG. 2. Although the array 30 is depicted as having four antennas, the array 30 can have a different number of antennas. Due to the curvature of the substrate 72, the upper surface 54a of the antenna 34a faces in the first direction along the axis A, and the upper surface 54b of the antenna 34b faces in a direction A′ that is slightly different than the direction of the axis A, but can be characterized as generally similar to the direction of the axis A. Similarly, the upper surface 56a of the antenna 36a faces in a direction along an axis B′ that is slightly different than the direction of the axis B of the upper surface 56b of the antenna 36b, but can be characterized as generally similar to the direction of the axis B. The axes A, A′ both diverge away from the axes B, B′ and vice versa. The substrate 72 may be formed in a manner so it does not interfere with operation of the antenna array 30 as transmit/receive antenna, for example being formed of a non-conductive material such as plastic.

Referring again to FIG. 1, the transmit antenna 11 can have any form suitable for transmitting the RF/microwave transmit signal 21. For example, the transmit antenna 11 can include a strip or patch of conductive material such as metal or other material that can transmit signals in the radio or microwave frequency range of the electromagnetic spectrum. The transmit antenna 11 may reside on the surface of a substrate or plate, the transmit antenna 11 may partially protrude from (i.e., be partially embedded within) the substrate or plate as depicted in FIGS. 2 and 3, or the transmit antenna 11 may be completely embedded within the substrate or plate. The substrate or plate may be formed in a manner so it does not interfere with operation of the transmit antenna 11, for example being formed of a non-conductive material such as plastic.

The receive antenna 13 can have any form suitable for receiving RF/microwave signals from the solid sample as a result of transmitting the transmit signal by the at least one transmit antenna to and/or into the solid sample. For example, the receive antenna 13 can include a strip or patch of conductive material such as metal or other material that can receive signals in the radio or microwave frequency range of the electromagnetic spectrum. The receive antenna 13 may reside on the surface of a substrate or plate, the receive antenna 13 may partially protrude from (i.e. be partially embedded within) the substrate or plate, or the receive antenna 13 may be completely embedded within the substrate or plate as depicted in FIGS. 2 and 3. The substrate or plate may be formed in a manner so it does not interfere with operation of the receive antenna 13, for example being formed of a non-conductive material such as plastic.

One or both of the antennas 11, 13 may be position adjustable in order to alter the relative position/orientation between the antennas 11, 13, between the transmit antenna 11 and the object surface 71, and between the receive antenna 13 and the object surface 71. For example, the substrate(s) for one or both of the antennas 11, 13 can be mounted to a movable/rotatable mechanism to adjust the relative position/orientation of the antennas 11, 13. Alternatively, the antennas 12, 14 may be position adjustable while keeping the plates 16, 18 stationary. Changing the relative position/orientation between the antennas 11, 13 may change the sensing performance of the solid constituent sensor system 5. In an embodiment, the angles and/or shapes of the antennas 11, 13 may be changed in order to alter the performance of the sensor system 5. An example of an RF constituent sensor with adjustable transmit and/or receive components is disclosed in U.S. Pat. No. 11,696,698, the entire contents of which are incorporated by reference.

FIG. 4 depicts an example of the signal storage 14 of FIG. 1 for storing multiple sample solid constituent reference signals 36. The signal storage 14 may store a single one of the reference signals 36. The reference signals 36 that are stored can be based on the purpose of detecting and characterizing the solid sample 7 of FIG. 1. Many signals 36 are possible. In an embodiment, each signal 36 can represent a different level of a particular constituent, such as carbon content, in the solid sample (e.g., an iron compound or alloy). In an embodiment, each signal 36 may represent a different total amount of a combination of multiple constituents in the solid samples. In an embodiment, each signal 36 can represent a different molecular makeup of homogenous or heterogeneous solid samples. In an embodiment, each signal 36 can represent a different type of metal (e.g. gold, silver, platinum, etc.) for use in identifying the solid sample 7. Different signal sets may be stored. For example, one set of stored reference signals 36 can be provided for one type of constituent in the solid sample, and a second set of stored reference signals 36 can be provided for a second type of constituent in the solid sample.

The reference signals 36 may be stored in a database in the signal storage 14. The database may include one or more database tables, of which every column of a database table represents a particular variable or field, and each row/column of the database table may correspond to a given record of the database. The database tables may list values for each of the variables, and/or for each record of the dataset. Various signal forms of various solid samples can be stored and organized in the database tables.

Referring again to FIG. 1, the controller 19 may be part of the transmit circuit 15, part of the receive circuit 17, or the transmit circuit 15 may include a controller that is separate from a controller included in the receive circuit 17. The solid constituent sensor system 5 can also include a power supply, such as a battery (not shown) which may or may not be rechargeable. In some embodiments, power can be provided from mains power, for example by plugging the solid constituent sensor system 5 into a wall socket via a cord connected to the solid constituent sensor 10, or by another external power source.

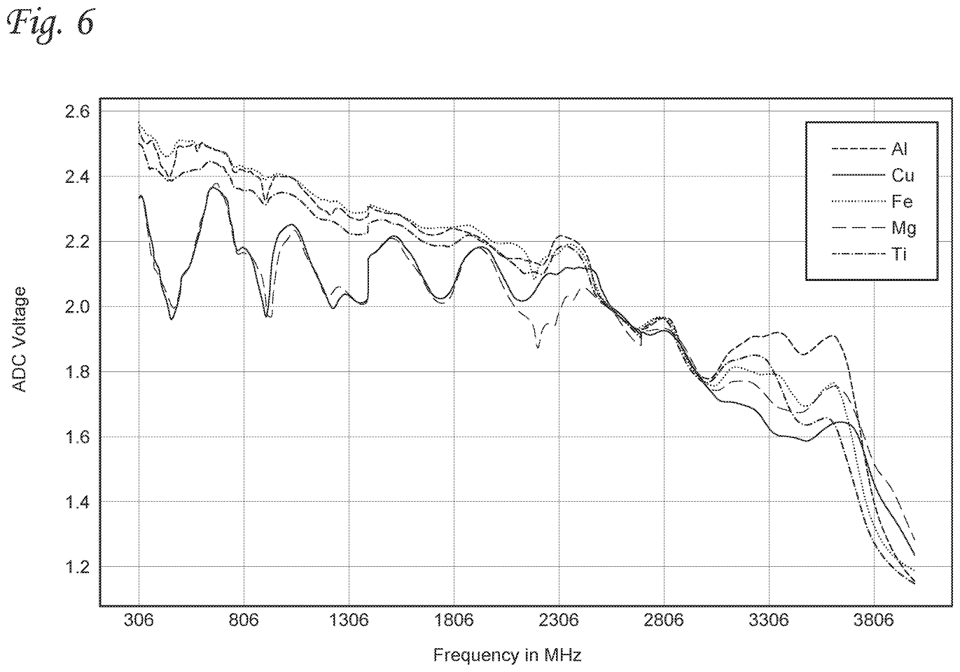

Referring to FIG. 5, a flow chart 50 illustrating an example of a solid constituent detection and characterization method, for example using the sensor system 5 described herein, is illustrated, in accordance with at least some embodiments described herein. The processing flow 50 can include one or more operations, actions, or functions as illustrated by one or more of blocks 52, 54 and 56. These various operations, functions, or actions may, for example, correspond to software, program code, or program instructions executable by a processor that causes the functions to be performed. Although illustrated as discrete blocks, obvious modifications may be made, e.g., two or more of the steps or blocks may be re-ordered; further steps or blocks may be added; and various steps or blocks may be divided into additional steps or blocks, combined into fewer steps or blocks, or eliminated, depending on the desired implementation. Further steps or blocks not illustrated in FIG. 5 may be included. Processing flow 50 may begin at block 52.

At block 52 (Transmit RF/microwave signal into solid sample), a transmit signal that is in a radio or microwave frequency range of the electromagnetic spectrum is transmitted from the transmit antenna to and/or into the solid sample. The transmit signal may be a single discrete signal or the signal may be part of a frequency sweep in which multiple signals are transmitted. The frequency sweep may start at an initial frequency and end at a final frequency, with the signals separated by equal or unequal frequency steps. An example of performing frequency sweeps in a radio/microwave frequency sensor is disclosed in U.S. Pat. No. 11,033,208, the entire contents of which are incorporated herein by reference. Processing may proceed from block 52 to block 54.

At block 54 (Detect response from the solid sample), a response is detected at the receive antenna. The response results from transmission of the transmit signal to and/or into the solid sample. The transmitted signal may interact with the solid sample and constituents thereof as it transmits to and/or into the solid sample. The interaction may alter the transmit signal and generate one or more electromagnetic response signals which can be detected by the receive antenna 13 and used to characterize the solid sample. Processing may proceed from block 54 to block 56.

At block 56 (Characterize the solid sample based on the response), the detected response is then used to characterize the solid sample. For example, the detected signal waveform may, in its original form or after being converted to other suitable forms by a processor of the sensor system 5, be compared to one or more stored reference signals as described above for FIG. 4.

In an embodiment, the detected signal may be compared to a single reference signal. When the detected signal deviates from the reference signal, the sensor system 5 can determine that the detected solid sample deviates from an expected chemical composition, and a notification/alarm signal can be sent, for example to an alarm, to notify a user of a related problem of the detected solid sample. The detected signal may need to deviate from the stored signal by a predetermined amount, such as 10% or more, 5% or more, 3% or more, or other quantifiable amount in order to generate a notification signal. The deviation of the detected signal from the stored reference signal can indicate a number of problems such as metal fatigue. The detected signal may also match the reference signal which can indicate that the solid sample has an expected chemical composition and can be used for material identification.

In another embodiment, the detected signal may be compared to multiple reference signals in an effort to look for a match between the detected signal and one of the reference signals. A match can indicate that the solid sample is acceptable or has an expected chemical composition, structure, or property, or a match can indicate that the solid sample is not acceptable or may not have an expected chemical composition, structure, or property.

The solid constituent sensors 5 described herein may form or be incorporated into residential, industrial and/or commercial sensors/detectors. Examples of sensors that the solid constituent sensor 10 can be incorporated into include, but are not limited to, a metal detector, an infrastructure detector, an aircraft parts detector, a soil detector, a plant detector, and many others.

In an embodiment, a metal detector may include one or more of the solid constituent sensors described herein. The one or more of the solid constituent sensors can detect one or more constituents in a solid metallic material and characterize the solid metallic material based on the detected constituents, including, for example, determining metal fatigue, water saturation, metal identification, etc. For example, metal identification can be determined using a chemical composition analysis based on the detected one or more constituents in the solid metallic material sample. The detected signal can be compared to one or more stored reference signals of known standards or reference databases to determine its identify. In an embodiment, metal fatigue can be assessed by analyzing the one or more detected constituents (e.g., by comparing to the stored reference signal(s)) to determine crystallographic defects, microstructure changes, and mechanical properties (e.g., hardness, brittleness, etc.) of the solid metallic material. In an embodiment, the one or more of the solid constituent sensors can also examine the presence or absence of water molecules or water-induced corrosion products (e.g., iron oxides, chlorides, sulfides, etc.) based on the detected signal and the stored reference signal(s).

In an embodiment, an infrastructure detector may include one or more of the solid constituent sensors described herein. The one or more of the solid constituent sensors can detect one or more constituents in a solid material of an infrastructure structure (e.g., a bridge part, a building part) and characterize the solid material based on the detected constituents.

In an embodiment, an aircraft parts detector may include one or more of the solid constituent sensors described herein. The one or more of the solid constituent sensors can detect one or more constituents in a solid material of an aircraft part (e.g., wings, fuselages, turbine blades, etc.) and characterize the solid material based on the detected constituents.

In an embodiment, a ship parts detector may include one or more of the solid constituent sensors described herein. The one or more of the solid constituent sensors can detect one or more constituents in a solid material of a ship part (e.g., hulls, propellers, etc.) and characterize the solid material based on the detected constituents.

In an embodiment, a soil detector may include one or more of the solid constituent sensors described herein. The one or more of the solid constituent sensors can detect one or more constituents in the soil and characterize the soil based on the detected constituents.

In an embodiment, a plant detector may include one or more of the solid constituent sensors described herein. The one or more of the solid constituent sensors can detect one or more constituents in plant and characterize/identify the plant based on the detected constituents.

### Example

In one example application the sensor described herein, the sensor can be used to non-invasively characterize and identify a metal object. Using the sensor, data from several thousand radio frequencies is collected and the results are passed to a random forest model to build a classifier. The model is then used to identify metal objects. For example, the metal objects can be metal cubes consisting of aluminum (Al), copper (Cu), iron (Fe), magnesium (Mg), and titanium (Ti).

As described above, the sensor generates RF signals and measures received power using an antenna array. For example, the sensor can generate RF signals that can range from about 300 MHz to about 4000 MHz. There is a transmit (Tx) amplifier to boost the signal, and then the RF signal is routed through a switch matrix that allows for it to be sent to any one of four antenna elements to be transmitted, for example as described in U.S. Pat. No. 11,058,331 which is incorporated herein by reference in its entirety. In this example, the antenna elements used were spaced 20 mm apart from one another. The antenna elements are mounted in a structure of milled stainless steel shielding to reduce RF interference from materials other than the metal object. The same switch matrix also establishes the receive (Rx) path, where one of the four antenna elements is chosen to receive the signal that results from the transmitted signal. Once through the switch matrix, the signal is amplified with a low noise amplifier (LNA) to set it in the appropriate range of the power measurement circuitry, which translates the received RF signal's power into a voltage output that can be sampled by the onboard analog to digital converter. The voltage output at each frequency depends on the dielectric properties of the metal object. Combining that information across thousands of frequencies shows each material's unique spectral response.

Initially, a training dataset is generated. Each metal cube was scanned 60 times using a protocol consisting of placing the cube on the sensor, running 10 frequency sweeps across the 300 MHz-4000 MHz range in 1 MHz steps, and then removing the cube. Each cube was thus placed on the sensor six times, with each frequency sweep consisting of 3701 frequencies. These 300 observations (60 scans×5 cubes) were used to train the model. After the model was trained, a ‘blind’ test was conducted. During this test, each of the metal cubes was scanned 10 times. The model then attempted to identify the metal cube independently given the results of each of the 50 scans in this test dataset.

Data analysis was carried out using Python 3.10 with the following packages and versions: numPy v1.22.4, pandas v1.5.3, matplotlib v3.7.1, and scikit-learn v1.2.2. The 300 observations described above were used as training data. For the purposes of model training, all sampled frequencies were treated as distinct features, and no effort at feature reduction was made. In this way, the model was built exclusively on “raw” data. The 300 observations×3701 frequencies=1,110,300 data points were passed to a Random Forest ML model, which was used to make classification predictions in the test set.

FIG. 6 shows the RF spectrum of each of the different metal cubes recorded by the sensor across a range of frequencies in the frequency sweep. Here, the x-axis represents RF frequency, and the y-axis represents the mean signal power recorded by the sensor (in terms of voltage) over all scans of the metal cubes. While the primary analysis and classification was performed using the Random Forest model, many of the features of these signals can be observed by eye. As shown in FIG. 6, each of the five metals displayed a unique spectral morphology, which in turn allowed the possibility of blind identification of the metal. Indeed, the Random Forest model built on the training dataset was able to identify the metal with 100% accuracy.

Based on a visual examination of FIG. 6, one area of interest for the purpose of identification is the 1900-2400 MHz range. The spectral responses of the 5 materials show a “trough” near 2200 MHz, at which more energy is absorbed by the metal material and less energy is therefore detected by the receive antenna. However, each metal is also unique in the precise frequency at which this occurs, and in the depth and width of the trough. The signals of the materials over the same region can also be compared quantitatively. With five cubes, there are 10 possible pairs of the five materials. Table 1 below shows the calculated L² distance between each pair, so a larger value means a greater difference between the signals.

TABLE 1
(Pairwise L² distances between the metal
cubes in the 1900-2400 MHz range)

| | Cu | Fe | Mg | Ti |
|---|---|---|---|---|
| Al | 1.8660 | 0.6710 | 3.6650 | 0.5430 |
| Cu | | 2.0270 | 2.0480 | 1.3890 |
| Fe | | | 3.6430 | 0.7800 |
| Mg | | | | 3.1500 |

Note that some of the metals seem to be “grouped” - aluminum, iron, and titanium have broadly similar responses in the 1900-2400 MHz range.

The examples disclosed in this application are to be considered in all respects as illustrative and not limitative. The scope of the invention is indicated by the appended claims rather than by the foregoing description; and all changes which come within the meaning and range of equivalency of the claims are intended to be embraced therein.

1. A solid constituent characterization method, comprising:
transmitting a transmit signal from a transmit antenna into a solid sample, the transmit signal is in a radio or microwave frequency range of the electromagnetic spectrum;
detecting a response at a receive antenna that results from transmission of the transmit signal to or into the solid sample; and
characterizing the solid sample based on the detected response.

2. The solid constituent characterization method of claim 1, wherein the solid sample includes one or more constituents within a solid material.

3. The solid constituent characterization method of claim 1, wherein the solid sample is a homogenous solid material or a heterogeneous solid material.

4. The solid constituent characterization method of claim 1, wherein the solid sample comprises a metallic alloy, a concrete material, or a soil.

5. The solid constituent characterization method of claim 1, further comprising boosting a power of the transmit signal before transmitting into the solid sample.

6. The solid constituent characterization method of claim 1, further comprising adjusting a penetration depth of the transmit signal transmitting to or into the solid sample.

7. The solid constituent characterization method of claim 1, wherein detecting the response comprises one or more electromagnetic response signals.

8. The solid constituent characterization method of claim 7, wherein characterizing the solid sample comprises comparing the one or more electromagnetic response signals with a stored reference signal.

9. The solid constituent characterization method of claim 7, wherein characterizing the solid sample comprises determining whether the one or more electromagnetic response signals match a stored reference signal or determining whether the one or more electromagnetic response signals deviate from the stored reference signal.

10. The solid constituent characterization method of claim 1, wherein characterizing the solid sample comprises determining presence of a constituent in the solid sample or determining absence of the constituent in the solid sample.

11. A solid constituent sensor system, comprising:
at least one transmit antenna and at least one receive antenna, the at least one transmit antenna is positioned and arranged to transmit a transmit signal to or into a solid sample, and the at least one receive antenna is positioned and arranged to detect a response resulting from transmission of the transmit signal by the at least one transmit antenna to or into the solid sample;
a power module to adjust a power of the transmit signal to adjust a penetration depth of the transmit signal transmitting to or into the solid sample;
a transmit circuit that is electrically connectable to the at least one transmit antenna, the transmit circuit is configured to generate the transmit signal in a radio or microwave frequency range of an electromagnetic spectrum;
a receive circuit that is electrically connectable to the at least one receive antenna, the receive circuit is configured to receive the response detected by the at least one receive antenna; and
a processor in communication with the receive circuit, the processor is configured to execute instructions to characterize the solid sample based on the detected response.

12. The solid constituent sensor system of claim 11, further comprises a data storage device that stores a plurality of reference solid constituent signals.

13. The solid constituent sensor system of claim 11, wherein the solid sample includes one or more constituents within a solid material.

14. The solid constituent sensor system of claim 11, wherein the processor to characterize the solid sample comprises the processor to compare the detected response with a stored reference signal.

15. The solid constituent sensor system of claim 11, wherein the processor to characterize the solid sample comprises the processor to determine whether the detected response matches a stored reference signal or determine whether the detected response deviates from the stored reference signal.

16. The solid constituent sensor system of claim 11, wherein the processor to characterize the solid sample comprises the processor to determine presence of a constituent in the solid sample or determining absence of the constituent in the solid sample.
